(19) United States
(12) Patent Application Publication
(10) Pub. No.: US 2003/0149437 A1
(43) Pub. Date: Aug. 7, 2003

(54) Methods of repairing longitudinal bone defects

(75) Inventors: Erella Livne, Haifa (IL); Samer Srouji, Nazareth (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Tcchnion Research & Development Foundation Ltd.

(21) Appl. No.: 10/377,648

(22) Filed: Mar. 4, 2003

Related U.S. Application Data
(63) Continuation-in-part of application No. PCT/IL01/00832, filed on Sep. 5, 2001.
(60) Provisional application No. 60/229,813, filed on Sep. 5, 2000.

(51) Int. Cl. A61F 2/28

(52) U.S. Cl. 606/76; 623/23.63; 435/397

(57) ABSTRACT

A method of repairing a long bone having a defect is provided. The method includes the steps of: (a) mechanically fixating the long bone or portions thereof; and (b) filling the defect with a biodegradable scaffold impregnated with growth factors and/or osteoprogenitor cells and awaiting for osteointegration to take place.

This application is a continuation-in-part of PCT/IL01/00832, filed Sep. 5, 2001, which claims the benefit of priority from U.S. patent application Ser. No. 09/713,037, filed Nov. 16, 2000 and from U.S. Provisional Patent Application No. 60/229,813, filed Sep. 5, 2000.

### FIELD AND BACKGROUND OF THE INVENTION

The present invention relates to methods of repairing longitudinal bone defects. More particularly, embodiments of the present invention relate to the use of hydrogel impregnated with bone growth promoting agents and/or osteoprogenitor cells for repairing bone defects such as, for example, fractures.

Bone healing, following for example, bone fractures, occurs in healthy individuals without a need for pharmacological and/or surgical intervention.

The bone healing process in an individual is effected by the physical condition and age thereof and by the severity of the injury and the type of bone injured.

Since improper bone healing can lead to severe pain, prolonged hospitalization and disabilities, cases in which a bone is severely damaged or in which the bone healing process in an individual is abnormal, oftentimes require external intervention, such as drug therapy, surgical implants or the like in order to ensure proper bone repair.

In cases where such external intervention is utilized for long bone or other skeletal bone repair, the repair must be sufficiently flexible so as to avoid repair induced bone damage, while being strong enough to withstand the forces subjected on the bone.

In many cases, especially those requiring bone defect repair, external intervention is typically effected using surgical implantation of organic or inorganic filler materials.

Numerous filler materials are known in the art. One example of a filler material is composed of autologous bone particles or segments which are removed from the patient and utilized directly for implantation. Although this type of filler material efficiently heals the defect, recovery and implantation thereof require long and costly surgical procedures.

Another typically used filler material is composed of hydroxyapatite obtained from sea coral or derived synthetically mixed with the patient's blood and/or bone marrow to form a gel or a putty. This material is osteoconductive but bioinert and as such it is absorbed into the natural bone, remaining in place indefinitely as a brittle, foreign body in the patient's tissue.

Allograft bone is also utilized as a filler material. Allograft bone is essentially a collagen fiber reinforced hydroxyapatite matrix containing active bone morphogenic proteins (BMPs), which can be provided in a sterile form. The demineralized form of allograft bone is naturally both osteoinductive and osteoconductive. The demineralized allograft bone tissue is fully incorporated in the patient's tissue and as such it has been used for many years in bone surgery to fill in bone defects.

Several attempts to enhance bone ingrowth around filler or implant have been described in recent years, see for example U.S. Pat. Nos. 4,928,959 and 5,046,484 and Legeros and Craig (1993) in the Journal of Bone and Mineral Research, vol. 8, Supplement 2 which describe methods for effecting integration of the implant into the endogenous bone.

Although these prior art documents suggest several methods of increasing implant or filler integration into the bone, they fail to teach an effective method of enhancing osteointegration and/or osteoinduction and implant stability.

Under normal conditions, the extracellular matrix in the bones and the cartilage is degraded and repaired constantly and in equal rate by the osteoclasts, osteoblasts and chondrocytes. These cells are responsible for synthesis and breakdown of cartilage and bone components, a process that is regulated by growth factors and cytokines. Reduction in these growth factors affects this process leading to bone diseases.

Insulin-like growth factor-1 (IGF-1) and transforming growth factor-β (TGF-β) are stored in the extracellular matrix of bones and cartilage. Such factors stimulate synthesis of collagen and proteoglycans in the extracellular matrix of the connective tissue. Proteoglycans are involved in matrix metabolism of normal cartilage and may play a role in matrix repair in patients suffering from diseases like osteoarthritis. Bone tissue is composed mainly of type I collagen, proteoglycans and various bone specific matrix macromolecules such as osteonectin and osteopontin.

Enzymes such as matrix metalloproteinases (MMPs) including collagenases, gelatinases stromelysin and tissue plasminogen activator also play a role in matrix metabolism. These degrading enzymes, which are synthesized by chondroblasts, osteoblasts and osteoclasts, participate in the degradation of the matrix, an activity, which is inhibited by endogenous inhibitors, such as tissue inhibitor metalloproteinases (TIMPs) also synthesized by bone and cartilage cells.

Various studies have also shown that TGF-β, IGF-1, bone morphogenic proteins (BMPs) (Lee et al., 1994; Gerhart et al., 1992) and basic fibroblast growth factor (bFGF) (Tabata et al., 1998) also participate in bone repair (Hong et al., 2000, Yamamoto et al., 2000, Moxham et al., 1996 and Toung et al., 1998).

Thus, growth factors are important mediators of bone regeneration. However, in vivo, these agents have a short life span in the matrix. Thus, researchers have directed their efforts towards increasing the availability of growth factors to the site of bone healing. One approach is to use scaffolding composed of guanidine-extracted demineralized bone matrix (Moxham et al., 1996), polymeric or ceramic implants (Gombotz et al., 1994), or bone grafts (Kenley et al., 1993) complexed with growth factors as an implant.

Recently, biodegradable hydrogels were shown to be a promising biomaterial matrix for growth factor release (Hong et al., 2000; Yamada et al., 1997; Yamamoto et al., 2000). It has been demonstrated that bFGF complexed with acid hydrogel has stimulatory effect on bone osteoinduction (Hong et al., 2000), that IGF-1 incorporated into type-I collagen gel enhanced nasal defects healing (Toung et al., 1998) and that TGF-β incorporated into acid gelatin hydrogel enhanced healing of rabbit skull defects.

However, none of these studies was aimed at orthopedic purposes and as such these studies did not address the effects of hydrogel mixed with growth factors such as, for example, TGF-β and IGF-1 or combinations thereof, on reconstruction of defects in long bones.

Osteoprogenitor cells are also known as important mediators of bone regeneration. The stromal compartment of the cavities of bone is composed of a net-like structure of interconnected mesenchymal cells. The role of the marrow stroma in creating the microenvironment for bone regeneration lies in a specific subpopulation of the stroma cells. The stroma cells differentiate from a common stem cell to the specific lineage, each of which has a different role. Their combined function results in orchestration of a 3-D-architecture that maintains the active bone marrow within the bone.

Usually, when bone marrow cells are cultivated in vitro, the vast majority of hemopoietic cells die and the cultures contain fibroblast-like adherent cells (MSF). When the cells are plated at low density, they are primarily composed of colonies of fibroblast-like morphology. The cells forming these colonies were described as colony fibroblastic unit-fibroblast (CFU-F). These cells, in a primary culture, are heterogeneous and the various fibroblastoid colonies differentiate to distinctive MSF cell types. Their distinct properties differ markedly: they contain subpopulations as fibroblasts, endothelial, adipocytes and osteogenic cells. The MSF cells differ in their capacity to form bone and/or to support the growth of hemopoietic (both lymphoid and myeloid) cell lines.

Since the formation of new bone matter is facilitated by osteogenic substances that induce progenitor cells in the surrounding bone, a therapeutic strategy that include administering precursor stem cells that are able to differentiate into bone cells is highly recommended. These cells are present at relatively low frequency in the marrow stroma, and their administration can stimulate the differentiation toward osteoblast lineage.

Several methods are known in the art to obtain osteoprogenitor cells. In one example, marrow stem cells were cultured in Dulbecco's modified Eagle's medium (DMEM) in the presence of 15% FCS, 2 mM L-glutamine, 50 U/ml penicillin, 50 μg/ml streptomycine, 50 μg/ml ascorbic acid, 50 nM beta-glycerophosphate, 10⁻⁷ M dexamethasone, retinoic acid or bFGF (Buttery et al., 2001).

The osteoprogenitor cells are characterized by an ability to form osteogenic nodules secreting Type-1 collagen and osteocalcin and an ability to induce mineralization of the surrounding matrix (Robinson and Nevo, 2001).

A similar approach has been used for directing the differentiation of embryonic stem cells to form osteoprogenitors, as reported by Thompson et al. (1998); Amit et al. (2000); Schuldiner et al. (2000) and Kehat et al. (2001).

However, none of these studies was aimed at orthopedic purposes and as such these studies did not address the effects of hydrogel mixed with osteoprogenitor cells on reconstruction of defects in long bones.

As has already been mentioned hereinabove, long bones are normally subject to, and operate against, substantial loads and forces.

In addition, in long bones, repair oftentimes necessitates reconstruction of bone portions of substantial length, a procedure that is not practiced in repair of other bones such as skull bones. As such, methods, which are employed for bone repair in general, including the methods utilizing the biodegradable hydrogels, described above, cannot be adapted or directly applied to long bone repair without a considerable amount of experimentation.

There is thus a widely recognized need for, and it would be highly advantageous to have, a method of repairing long bones devoid of the above limitation.

### SUMMARY OF THE INVENTION

While conceiving the present invention it was hypothesized that hydrogel impregnated with at least one bone growth-promoting agent, with cells expressing and secreting at least one growth factor and/or with osteoprogenitor cells will be beneficial for repairing longitudinal bone defects including fractures and will enhance the bone reconstruction.

While reducing the present invention to practice, a rat tibia defect model was used to assess the efficiency of hydrogels containing TGF-β, r IGF-1, osteoprogenitor cells or combinations thereof in enhancement of bone defect repair.

Thus, according to one aspect of the present invention there is provided a method of repairing a long bone having a defect. The method comprising (a) mechanically fixating the long bone or portions thereof; and (b) filling the defect with a biodegradable hydrogel designed to promote osteointegration and awaiting for osteointegration to take place. The method according to this aspect of the present invention is preferably effected by using an external mechanical fixating device, preferably as a sole fixating device, for fixating the long bone, and the long bone is preferably selected from the group consisting of a femur, tibia, humerus and a radius. The biodegradable hydrogel is preferably a cross-linked acidic gelatin.

According to another aspect of the present invention, there is provided another method of repairing a long bone having a defect. The method comprising (a) mechanically fixating the long bone or portions thereof; and (b) filling the defect with a biodegradable hydrogel containing osteoprogenitor cells and awaiting for osteointegration to take place. Mechanically fixating the long bone is preferably effected by an external fixating device. The biodegradable hydrogel is preferably a cross-linked polymer that comprises an acidic protein, preferably acidic gelatin.

According to further features in preferred embodiments of the invention described below, each of the methods described hereinabove further comprising reshaping the defect prior to fixating the long bone or to filling the defect.

According to still further features in the described preferred embodiments (a) precedes (b) or alternatively (b) precedes (a).

According to still further features in the described preferred embodiments each the described methods further comprises unfixating the long bone or portions thereof following osteointegration.

According to still another aspect of the present invention there is provided a kit for repairing a long bone having a defect, the kit comprising: (a) a mechanical fixating device for fixating the long bone or portions thereof; and (b) a filler for filling the defect, said filler including a biodegradable cross-linked acidic gelatin designed to promote osteointegration. The mechanical fixating device, according to this aspect of the present invention is preferably an external mechanical fixating device and the long bone is preferably selected from the group consisting of a femur, a tibia, a humerus and a radius.

According to yet another aspect of the present invention there is provided a kit for repairing a long bone having a defect, the kit comprising: (a) a mechanical fixating device for fixating the long bone or portions thereof; and (b) a filler for filling the defect, the filler including a biodegradable hydrogel containing osteoprogenitor cells. The mechanical fixating device is preferably an external mechanical fixating device. The biodegradable hydrogel is preferably a cross-linked polymer that comprises an acidic protein, preferably an acidic gelatin.

According to still further features in the described preferred embodiments the external mechanical fixating device is a cast or a bone securing device.

According to still further features in the described preferred embodiments the biodegradable hydrogel includes at least one bone growth-promoting agent impregnated therein or attached to the polymer.

According to still further features in the described preferred embodiments the biodegradable cross-linked acidic gelatin includes at least one bone growth-promoting agent impregnated therein or attached thereto.

According to still further features in the described preferred embodiments the at least one bone growth promoting agent is selected from the group consisting of an insulin-like growth, factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

According to still further features in the described preferred embodiments the at least one bone growth promoting agent is at least one cell type expressing and secreting at least one growth factor.

According to still further features in the described preferred embodiments the at least one growth factor is selected from the group consisting of an insulin-like growth factor-1 (IGF-1), a transforming growth factory-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

According to still further features in the described preferred embodiments the biodegradable cross-linked acidic gelatin includes osteoprogenitor cells alone or in combination with the at least one bone growth-promoting agent.

According to still further features in the described preferred embodiments the osteoprogenitor cells are impregnated in or attached to the cross-linked acidic gelatin.

According to still further features in the described preferred embodiments the osteoprogenitor cells comprise embryonic stem cells.

According to still further features in the described preferred embodiments the biodegradable hydrogel includes at least one drug impregnated therein or attached to the polymer.

According to still further features in the described preferred embodiments the biodegradable cross-linked acidic gelatin includes at least one drug impregnated therein or attached thereto.

According to still further features in the described preferred embodiments the at least one drug is selected from the group consisting of an antibiotic agent, a vitamin and an anti-inflammatory agent.

According to still further features in the described preferred embodiments the antibiotic is selected from the group consisting of an aminoglycoside, a penicillin, a cephalosporin, a semi-synthetic penicillin, and a quinoline.

According to still further features in the described preferred embodiments the long bone is selected from the group consisting of tibia, fibula, femur, humerus, radius, ulna, carpals, metacarpals, phalanges, tarsals, and metatarsals.

According to still further features in the described preferred embodiments the biodegradable hydrogel and the biodegradable cross-linked acidic gelatin both have electrostatic binding properties.

According to still further features in the described preferred embodiments the biodegradable hydrogel includes at least one bone degradation inhibitor impregnated therein or attached to the polymer.

According to still further features in the described preferred embodiments the biodegradable cross-linked acidic gelatin includes at least one bone degradation inhibitor impregnated therein or attached thereto.

According to still further features in the described preferred embodiments the at least one bone degradation inhibitor is selected from the group consisting of a tissue inhibitor metalloproteinases (TIMP) a collagenase inhibitor, a gelatinase inhibitor, a stromelysin inhibitor and a plasminogen activator inhibitor (PAI).

According to still further features in the described preferred embodiments the biodegradable hydrogel and the biodegradable cross-linked acidic gelatin are both adapted for sustained release of a therapeutically active agent.

According to still further features in the described preferred embodiments the defect is a result of a condition selected from the group consisting of a traumatic injury, surgery, osteotomy, malignant tumors, fracture malunion, fracture malformation, a birth defect, a developmental defect, aging and a disease.

The present invention successfully addresses the shortcomings of the presently known configurations by providing a biodegradable scaffold having osteoinductive and osteoconductive properties.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates the external fixation device of the present invention and the bone defect treated by the method of the present invention as photographed on the day of surgery.

FIGS. 2A-D illustrate the effects of hydrogel containing TGF-β, on the bone defect at the day of surgery (A), two weeks post surgery (B), four weeks post surgery (C) and six weeks post surgery (D).

FIGS. 3A-D illustrate the effects of non-loaded hydrogel on the bone defect at the day of operation (A), two weeks post surgery (B), four weeks post surgery (C) and six weeks post surgery (D).

FIGS. 4A-D illustrate the effects of saline loaded hydrogel on the bone defect at the day of surgery (A), two weeks post surgery (B), four weeks post surgery (C) and six weeks post surgery (D).

FIGS. 5A-D illustrate the effects of hydrogel loaded with IGF-1, on the bone defect at the day of surgery (A), two weeks post surgery (B), four weeks post surgery (C) and six weeks post surgery (D).

FIGS. 6A-D illustrate the effects of hydrogel loaded with TGF-β and IGF-1, on the bone defect at the day of surgery (A), two weeks post surgery (B), four weeks post surgery (C) and six weeks post surgery (D).

FIG. 7 illustrates a three dimensional (3-D) CT of bone defect in rat tibia on the day of surgery.

FIG. 8 is a 3-D CT illustrating the bone defects in the rat tibia, six weeks post treatment with hydrogel loaded with TGF-β (in the middle), IGF-1 (on the right) as compared to an untreated tibia (on the left).

FIG. 9 is a 3-D CT illustrating the bone defects in the rat tibia, six weeks post treatment with a hydrogel loaded with TGF-β and IGF-1 (on the left), not-loaded (in the middle) or loaded with saline (on the right).

### DESCRIPTION OF THE PREFERRED EMBODIMENTS

The present invention is of methods and kits, which can be used for promoting bone growth especially in long bones. Specifically, the present invention relates to a bone repair scaffold including gelatin hydrogel impregnated with bone growth promoting agents, such as, for example, TGF-β and/or IGF-1 and/or with osteoprogenitor cells, which are slowly released from the scaffold.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Although the prior art teaches of methods which are suitable for repairing bones, these methods are typically not suitable for repairing long bones such as the femur, tibia, humerus, radius ulna and the like, since specific problems which are unique to long bone repair are not addressed thereby.

As is further exemplified in the Examples section, which follows, the present inventors have developed a bone repair kit and method, which are optimized for long bone repair.

The present invention enables to restore mechanical, architectural and structural competence to bones having defects, while providing structural surface areas, which can serve as efficient substrates for the biological process governing bone and soft tissue healing and regeneration.

In addition, the method of the present invention generates an electronegative environment within the treated bone wound, by electrokinetic and electrochemical means, thus leading to the formation of an acidic environment which is conductive to osteogenesis.

Furthermore, the present invention considerably accelerates the cellular and biological processes involved in bone repair, a feature that is extremely important for long bone repair, especially long bones which are constantly subjected to considerable forces.

The principles and operation of the present invention may be better understood with reference to the drawings and accompanying descriptions.

As used hereinafter the phrase “long bone” refers to a bone having a length, which is at least two times longer than the diameter. Typically, the phrase “long bone” refers to the bones of the extremities, i.e. the tibia, fibula, femur, humerus, radius, ulna, carpals, metacarpals, phalanges, tarsals and metatarsals. More specifically, the phrase “long bone”, as used herein, refers to the four main bones of extremities, i.e., the tibia, the femur, the humerus and the radius.

Hence, in one embodiment, the method and kit of the present invention are directed toward repairing a tibia, a femur, a humerus and/or a radius bone having a bone defect.

As used hereinabove the term “bone defect”, refers to any abnormality in the bone, including, but not limited to, a void, a cavity, a conformational discontinuity, a fracture or any structural change produced by injury, osteotomy, surgery, fractures, malformed healing, non-union fractures, skeletal deformations, aging, or disease.

According to one aspect of the present invention there is provided a kit for repairing a long bone having a defect. The kit includes a mechanical fixating device for fixating the long bone or portions thereof. Examples of a fixation device include, but are not limited to, flanges, rods, bars, wires, staples, screws, sutures as well as various casts, sleeves and the like, which are typically external fixation devices. Hence, the kit of the present invention preferably includes an external mechanical fixating device. The kit further includes a filler for filling the defect, which includes a biodegradable hydrogel designed to promote osteointegration. The biodegradable hydrogel is preferably a cross-linked acidic gelatin. Alternatively or additionally, the biodegradable hydrogel includes osteoprogenitor cells, as is detailed hereinbelow.

The kit according to this aspect of the present invention is utilized for long bone repair.

As such, according to another aspect of the present invention there is provided a method of treating long bone defects, such as fractures, cavities voids and the like.

The method according to the present invention is effected by mechanically fixating the long bone or portions thereof prior to, or following a step of filling the defect with a biodegradable scaffold.

Preferably, the method further includes the step of reshaping the defect prior to bone fixating or defect filling. Such reshaping can be performed with, for example, drills or grinders or any other device utilizable for reshaping the defect.

It will be appreciated that the type of fixating device utilized in the step of fixating depends on the type of defect to be repaired and on the type and placement of the bone. Preferably, the fixating device is an external fixating device. More preferably, an external fixating device such as, for example, a cast, is the only fixating device utilized in the method of the present invention, accompanied with an injection of the filer material.

However, it should be noted in this respect that in cases that necessitate a fully invasive surgical procedure, an internal fixating device can be utilized as well.

Following a time period required for osteointegration, which varies according to the type of repair, condition of the patient and the like, the fixating device can be removed.

The use of a biodegradable scaffold as a filler base is of important advantages.

Similar to non-degradable scaffolds described in the art, the biodegradable scaffold enables restoration of mechanical, architectural and structural competence to the bone void treated, provide a stable surface structure for the genesis, the growth and the development of calcified and non calcified connective tissue, and acts as a carrier to the drugs.

However, in contrast to non-degradable scaffolds, the use of a completely biodegradable scaffold as an implant is advantageous in that it traverses the need for a second surgery in order to remove the implant.

In addition, the biodegradation process enables the release of scaffold attached biologically active agents, such as enzymes, proteins, antibiotics, vitamins, cells or growth factors, which are described in greater detail below and in the Examples section which follows.

Various biologically active agents can be directly or indirectly attached to the scaffold via chemical reactions known in the art.

Preferably, the biodegradable scaffold includes a cross-linked polymer such as a protein or a polysaccharide, which functions as a carrier for biologically active agents. Preferably, the polymer is an acidic protein such as, but not limited to, acidic gelatin.

According to another preferred embodiment of the present invention, the scaffold includes charged or polar groups, which are either introduced in the scaffold fabrication process or attached to the scaffold following fabrication. In any case, such groups, which are preferably negatively charged, enable the binding of positively charged substances such as growth factors. In addition, the negatively charged scaffold creates an acidic, electronegative environment, which is inductive and conducive to osteogenesis.

According to another preferred embodiment of the present invention the biodegradable scaffold is fabricated from a hydrogel. Preparation, sterilization and loading of hydrogel is described in detail in the Examples section below.

The hydrogel can be loaded with various substances including growth factors, such as but not limited to, insulin-like growth factor-1 (IGF-1), transforming growth factory-β(TGF-β), basic fibroblast growth factor (bFGF), bone morphogenic proteins (BMPs) such as, for example, BMP-2 or BMP-7, cartilage-inducing factor-A, cartilage-inducing factor-B, osteoid-inducing factor, collagen growth factor and osteogenin. In general, TGF plays a central role in regulating tissue healing by affecting cell proliferation, gene expression and matrix protein synthesis, BMP initiates gene expression which leads to cell replication, and BDGF is an agent that increases activity of already active genes in order to accelerate the rate of cellular replication. All the above-described growth factors may be isolated from a natural source (e.g., mammalian tissue) or they may be produced as recombinant peptides.

The hydrogel can be also loaded with cell types that express and secrete the growth factors described hereinabove. These cells include cells that produce growth factors and induce their translocation from a cytoplasmic location to a non-cytoplasmic location. Such cells include cells that naturally express and secrete the growth factors or cells which are genetically modified to express and secrete the growth factors. Such cells are well known in the art.

Recent studies have demonstrated that biodegradable hydrogel is highly suitable for use as a biomaterial matrix for growth factors release. These studies demonstrated that the disappearance of hydrogel from a defect was due to replacement with bone mineral deposition (Yamamoto et al., 2000) conclusively showing that hydrogel does not interfere with the process of bone formation.

As mentioned hereinabove, the scaffold includes biologically active agents, which are attached directly or indirectly thereto. Such agents can be enzymes, and various growth factors, which participate in the bone replacement process, or they can be enzymes such as MMPs, which participate in hydrogel degradation.

Various growth factors can be attached to or impregnated in the hydrogel scaffold. For example, TGF-β is a growth factor capable of recruiting activated cells present around the bone defect. Such cells are capable of synthesizing enzymes, such as MMPs, which degrade the hydrogel.

Several examples of hydrogels containing growth factors such as, TGF-β (Yamamoto et al., 2000) and bFGF (Tabata et al., 1999) are known in the art.

Studies utilizing such hydrogels demonstrated that tissue response to growth factors released from such hydrogels was first detected eight weeks post surgery (Lee et al., 1994). As is further detailed in the Examples section which follows, the method of the present invention produces a response as early as four to six weeks following surgery thus considerably shortening the response time as compared to the prior art. It will be appreciated that this feature of the method of the present invention is extremely important since it enables faster recovery in bones, which are crucial for locomotion and other physical activities.

The hydrogel of the present invention can also be loaded with osteoprogenitor cells. Osteoprogenitor cells, as is known in the art, include an osteogenic subpopulation of the marrow stromal cells, characterized as bone forming cells. The osteoprogenitor cells utilized by the method of the present invention can include osteogenic bone forming cells per se and/or embryonic stem cells that form osteoprogenitor cells. The osteoprogenitor cells can be isolated using known procedures, as described hereinabove in the Background section or in Buttery et al. (2001), Thompson et al. (1998), Amit et al. (2000), Schuldiner et al. (2000) and Kehat et al. (2001). Such cells are preferably of an autological source and include, for example, human embryonic stem cells, murine or human osteoprogenitor cells, murine or human osteoprogenitor marrow-derived cells, murine or human osteoprogenitor embryonic-derived cells and murine or human embryonic cells. These cells can further serve as cells secreting growth factors, as described by Robinson and Nevo (2001), which are defined hereinabove.

The hydrogel of the present invention can thus be loaded with various active therapeutic agents, which can include bone growth-promoting agents, osteoprogenitor cells or a combination thereof. The scaffold utilized by the method of the present invention can also include at least one drug, such as, a vitamin, an antibiotic, an anti-inflammatory agent and the like which can be either impregnated into the hydrogel matrix or attached directly or indirectly (via a polymer) thereto.

Examples of suitable antibiotic drugs which can be utilized with the present invention include, for example, antibiotics from the aminoglycoside, penicillin, cephalosporin, semi-synthetic penicillins, and quinoline classes.

Preferably, the present invention utilizes an antibiotic or a combination of antibiotics which cover a wide range of bacterial infections typical of bone or surrounding tissue. Preferably, of these antibiotics types which are also efficiently released from, the scaffold are selected.

Vitamins such as, for example, vitamin D, ergocalciferol (vitamin D₂), cholecalciferol (vitamin D₃) and their biologically active metabolites and precursors can be utilized by the present invention.

Anti-inflammatory agents may be used in the present invention to treat or prevent inflammation and pain in the treated and surrounding area following treatment. The preferred anti-inflammatory agents are without limitation, indomethacin, etodolac, diclofenac, ibuprofen, naproxen and the like.

Other drugs, which may be beneficial to the present invention, include amino acids, peptides, co-factors for protein synthesis anti-tumor agent, immunosuppressants and the like.

As already mentioned hereinabove, under normal conditions, the extracellular matrix of bones and cartilage is degraded and repaired constantly and in equal rate by osteoclasts and degrading enzymes such as MMPs. As such, the biodegradable scaffold utilized by the present invention can include at least one bone degradation inhibitor such as, for example, a collagenase inhibitor, a gelatinase inhibitor, a stromeylsin inhibitor or a plasminogen activator inhibitor.

Thus, the present invention provides kits and methods utilizing same, which can be utilized to repair bone defects in long bones. By utilizing a biodegradable scaffold as a filler base along with attached or impregnated growth factors, osteoprogenitor cells and/or enzymes, the present invention ensures faster and better recovery of the bone defects.

Since the present invention ensures that scaffold degradation and new bone formation are inter-dependent, scaffold degradation which is too slow and which can lead to rapid elimination of growth factors and ingrowth of soft tissue at the defect site, or scaffold degradation which is too slow and as such impedes new bone formation (Yamamoto et al., 2000) are eliminated.

As is evident from experiments conducted while reducing the present invention to practice (see the Examples section which follows), the hydrogel scaffold of the present invention was completely degraded two weeks following surgery, at which point the newly formed bone appeared to be spongy, a clear indication of extensive bone formation.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include biochemical techniques. Such techniques are thoroughly explained in the literature. See, for example, “Cell Biology: A Laboratory Handbook”, Volumes I-III Cellis, J. E., ed. (1994); “Animal Cell Culture” Freshney, R. I., ed. (1986); “Immobilized Cells and Enzymes” IRL Press, (1986); and “Methods in Enzymology” Vol. 1-317, Academic Press; all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

While reducing the present invention to practice, the ability of TGF-β and IGF-1 incorporated into acidic gelatin hydrogel to induce bone regeneration in a rat tibia defect model was tested. Segmental bone defects (4 mm) were induced in the right tibiae of Sprague-Dawley rats by micrometer. External fixation was performed prior to induction of the bone defect. Hydrogel (95%, wt) either alone or mixed with any of the following treatments; TGF-β (0.1 μg), IGF-1 (25 ng), TGF-β (0.1 μg)+IGF-1 (25 ng) or saline, was inserted into the bone defect. The effect of the gelatin hydrogel was assessed by radiological soft tissue X-rays and computerized topography (CT) scan and three-dimensional (3-D) CT scan. At the end of the experiment the tibiae were dissected and their morphology studied.

Experimental Procedures:

Hydrogel Preparation:

Hydrogel (95% wt) was prepared by chemically crosslinking a 10% aqueous acidic gelatin (Nitta Gelatin Co. Osaka, Japan) solution with 5.0 mM glutaraldehyde at 4° C. The acidic gelatin, which was isolated from bovine bone using an alkaline process, is a 99 kDa molecule with an isoelectric point of 5.0; the gelatin was designated acidic because of its electrostatic ability,

The mixed acidic gelatin and glutaraldehyde hydrogel was cast into plastic molds (4×4×4 mm). The crosslinking reaction was allowed to proceed for 24 h at 4° C. following which the cross linked hydrogel was immersed in 50 mM glycine aqueous solution at 37° C. for 1 h to block residual aldehyde groups of glutaraldehyde. The resulting hydrogel was punched out and rinsed by double distilled water (DDW), and 100% ethanol and finally autoclaved to obtain sterilized hydrogel. The sterilized hydrogel were aseptically freeze dried (1 hour), and the water content was calculated in percent by weighing the hydrogel prior to, and following freeze drying.

Impregnation of the Growth Factor into the Hydrogel:

Impregnation of TGF-β (0.1 μg), IGF-1 (25 ng) or saline was carried out by immersing each freeze dried hydrogel in 600 μl of impregnating solution overnight at 4° C. and the swollen hydrogel was used for the various experimental groups. A similar procedure was used for impregnation of IGF-1 and TGF-β+IGF-1 into acidic gelatin hydrogel.

The hydrogel was also weighed prior to and following the swelling process.

Isolation and Integration of Osteoprogenitor Cells in the Hydrogel:

Osteoprogenitor cells are obtained according to the method described by Buttery et al. (2001) and characterized according to the methods described by Robinson and Nevo (2001). Human embryonic stem cells are similarly cultured in the presence of 15% FCS, 2 mM L-glutamine, 50 U/ml penicillin, 50 μg/ml streptomycine, 50 μg/ml ascorbic acid, 50 nM β-glycerophosphate, 10⁻⁷ M dexamethasone, retinoic acid or bFGF. Nodules demonstrating the osteogenic activity, formed by the cultured cells, are tested for their ability to secrete Type-1 collagen and ostocalcin, using immunohistochemistry for demonstration of bone-specific proteins. Alizarin red, von Kossa staining and electron microscopy are used for demonstration of mineral deposits in the surrounding matrix of the nodules.

The osteoprogenitor cells and/or the embryonic stem cells are then collected and incorporated into the hydrogel, prepared as described hereinabove, using the procedure described hereinabove for impregnation of growth factors into the hydrogel.

Formation of Defects in the Rats' Tibiae:

Ten Sprauge-Dawley rats (300 grams, 3 month-old) were used in each experimental group of the present study. Animals were anaesthetized (Ketamine 15 mg/kg body weight) and segmental bone defects were performed in their right tibiae. Prior to the induction of bone defects, a rigid external fixation was achieved by insertion of two pins through the proximal and medial tibiae. The edges of the pins were inserted through threaded brass rods fitted with nuts on each side of the tibia thus building a rigid frame. Brass rods were 4.8 mm in diameter and 33 mm long. Each rod was cut longitudinally from both ends to an equal length of 13 mm. These cuts were 1.0-1.2 mm width in order to support the needles. The overall weight of the device was 12 grams. Complete transverse segmental bone defects (4 mm) were induced in the tibiae between the two inserted needles, using a micromotor drill of 4 mm in diameter (FIG. 1). The distance between the two inserted needles was 13 mm. The formed defect was filled with hydrogel, 95% wt, containing TGF-β (0.1 μg), IGF-1 (25 ng), TGF-β (0.1 μg)+IGF-1 (25 ng) or saline which was prepared as described above, following which, muscles, soft tissues and skin were carefully sutured.

Assessment of the Bone Regeneration:

Bone regeneration at the defect was assessed by soft tissue X-rays (7.5 mA; 0.5 sec) taken immediately following surgery and two, four and six weeks post surgery. Upon termination of the experiment, animals were sacrificed and lower limbs were dissected and collected for general morphology, computerized topography (CT) scan (65-80 kV; 20 sec) and three-dimensional (3-D) CT scan (Marconi, M×8,000). Tissues were then fixed in 10% neutral buffered formalin (NBF), decalcified in 10% ethylene diamine tetra-acetic acid (EDTA) in 0.1 M Tris-HCl, pH 7.4 (3 weeks), embedded in Paraplast (Sherwood Medical, Mo. USA), sectioned and stained with hematoxylin and eosin (H&E).

Experimental Results:

The right tibia of each animal was immobilized by external fixation device and complete bicortical segmental defect was induced. Gelatin hydrogel was inserted to fill the bone defect (FIG. 1). Soft tissue X-ray that was taken at the day of operation clearly revealed bone discontinuity (gap) between the two inserted needles.

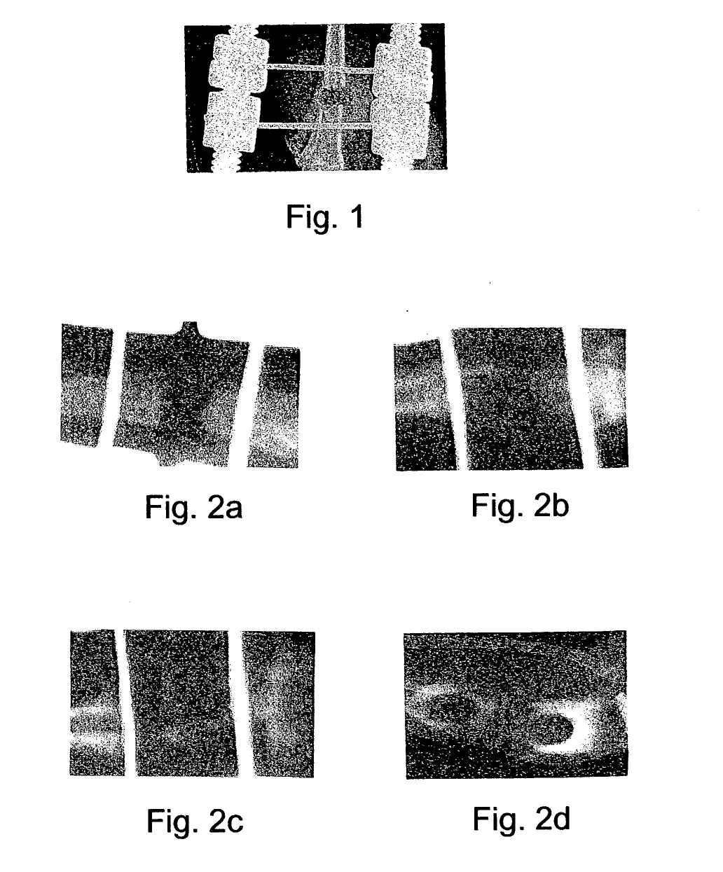

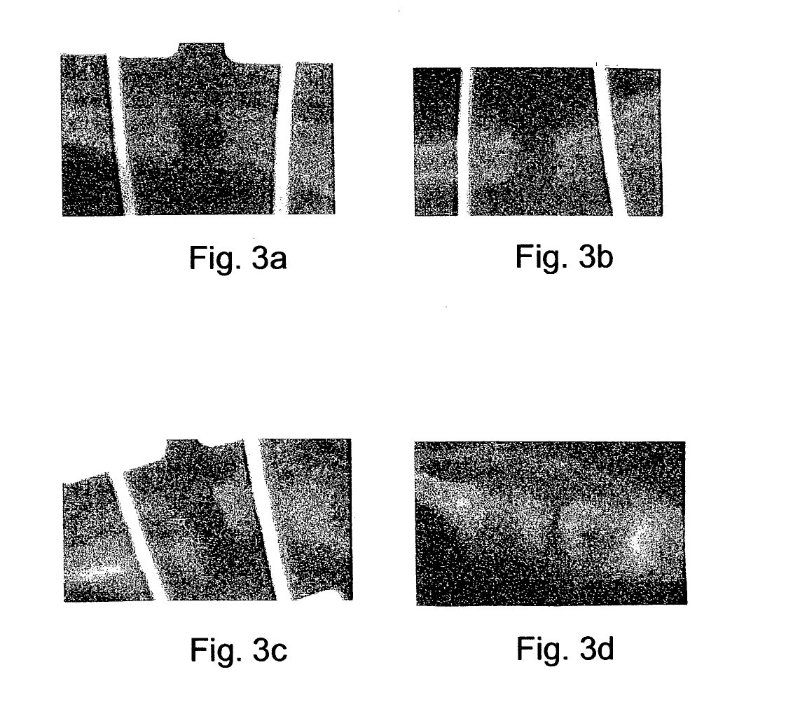

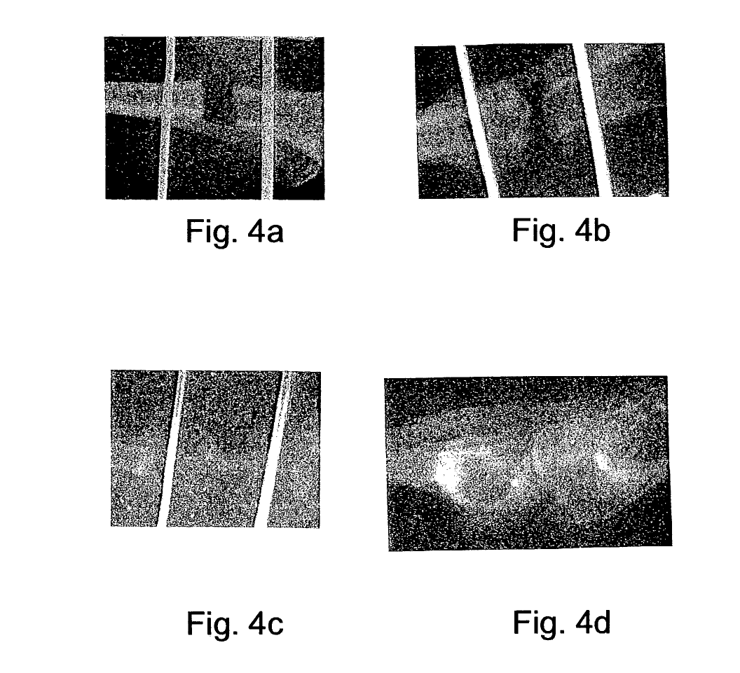

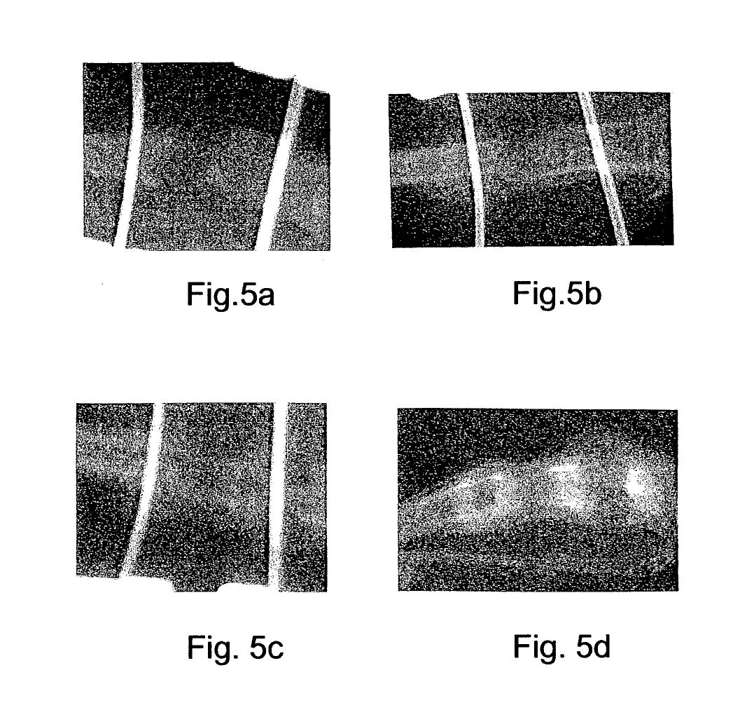

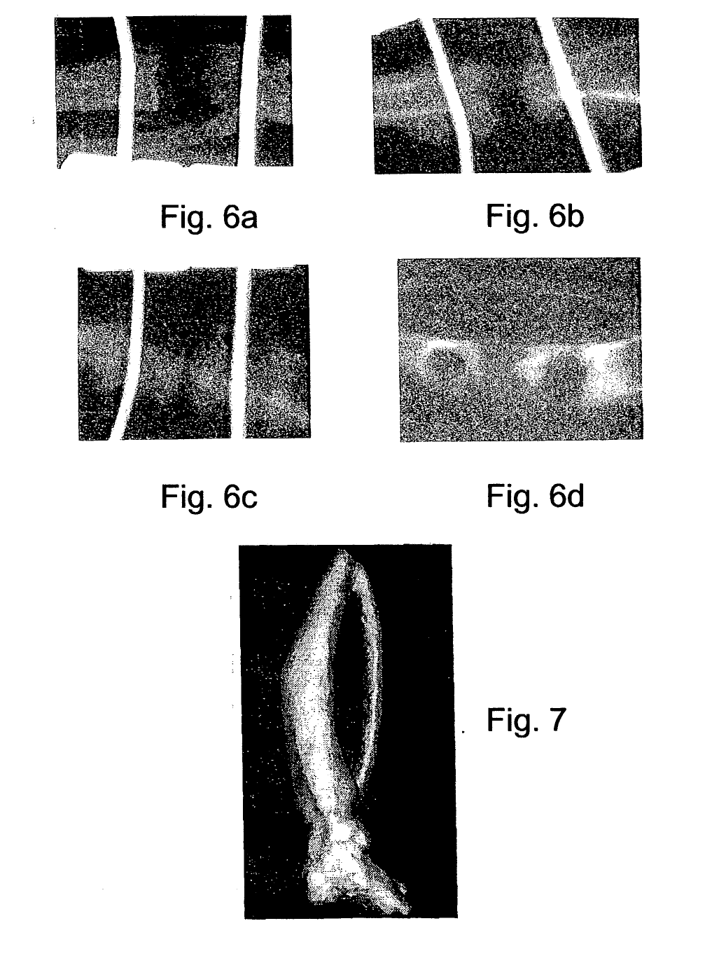

X-ray photographs that were performed as early as two weeks post operation revealed the presence of an opaque material between the incision boundaries of the defect in the TGF-β treated group (FIGS. 2B, 3B, 4B, 5B, 6B). The presence of such opaque material was not detected in rats treated with hydrogel or saline-containing hydrogel. Following four weeks, the amount of the calcified material observed by X-ray analysis increased in the TGF-β and in IGF-1 groups (FIGS. 2C, 3C, 4C, 5C, 6C). A solid bone bridge, which aligned with the line of the old cortices in the TGF-β, the IGF-1 and in the combination of TGF-β+IGF-1 treated groups (FIGS. 2D, 3D, 4D, 5D, 6D) was observed. Complete bone induction and newly formed cortices were observed in the area previously including the defect. The largest increase in calcified material was observed in the rats treated with a combination of TGF-β+IGF-1 (FIG. 6). In this group, the bone defect completely filled with newly formed bone six weeks following treatment.

Bone formation was observed around the external fixation needles and the external fixation device of the growth factor treated animals. This bone formation appeared to be rigid and firmly attached throughout the experimental period. In saline containing hydrogel or hydrogel alone this area appeared to be less calcified and the external fixation appeared to be loose (FIG. 2D).

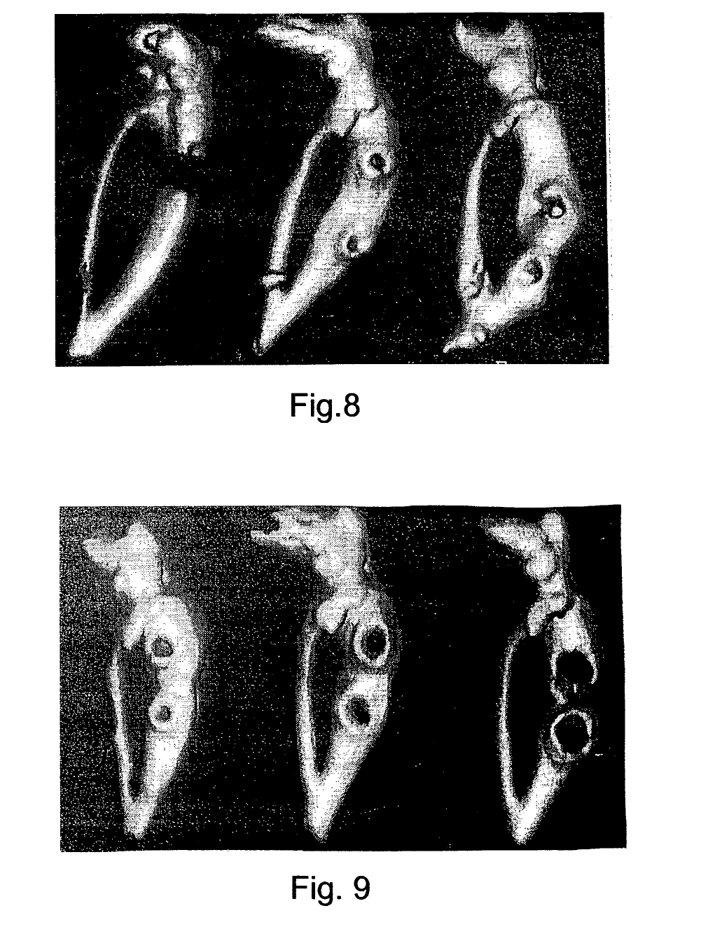

Axial CT revealed strong radiopacity at the site of the bone defect only in the TGF-β, IGF-1 or TGF-β+IGF-1 treated groups and not in the non treated (i.e., hydrogel alone and saline-containing hydrogel) groups. In the TGF-β, IGF-1 or TGF-β+IGF-1 treated groups the radiopacity of the injured tibiae was comparable with the adjacent fibulae, whereas in the hydrogel or saline-containing hydrogel treated animals, the radiological appearance of the tibia and the fibula was completely different. 3-D CT revealed that bones treated with TGF-β, IGF-1 or TGF-β+IGF-1 have completely restored their three dimensional shape and appeared to be even thicker than non-treated bones, whereas in bones treated with hydrogel or saline-containing hydrogel, a narrow cleft was still present in the defect region even six weeks post treatment (FIGS. 7-9).

The above example clearly demonstrates that gelatin hydrogel can serve as a good osteoconductive matrix for growth factors while still providing space for bone regeneration. A gelatin hydrogel mixed with, for example, TGF-β and/or IGF-1 is thus a promising surgical tool for enhancement of osteoinduction and osteointegration in bone defects.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### CITED REFERENCES

Amit, M., Carpenter, M. K., Inokuma M. S., Chiu, C-P, Harris, C. P., Watkins, M. A., Itskovitz-Eldor, J. and Thomson J. A. Clonally derived human embryonic stem cell lines maintain pluripotency and proliferative potential for prolonged periods o culture. Dev. Biol. 227-1-8, 2000.

Bosch, C., Melsen, B., Gibbons, R. Human recombinant transforming growth factor-β1 in healing of calvarial bone defects. J.Craniofac.Surg. 7:300-310;1996

Buttery, L. D. K., Bourne, S., Xynos, J. D., Wood, H., Hughes, B. D. S, Hughes, S. P. F., Episkopou, V., and Polak. Differentiation of osteoblasts and bone formation from murine embryonic stem cells. Tiss. Eng. 7: 89-99, 2001

Cook, S. D., Wolfe, M. W., Salkeld, S. L. Effect of recombinant human osteogenic protein-1 on healing of segmental defects in non-human primates. J.Bone Jt. Surg. 77-A:734-750;1995

Gerhart, T. N., Kirker-Head, C. A., Kriz, M. J., Holtrop, M. E., Henning, G. E., Hipp, J., Schelling, S. H., and Wang, E. Healing segmental femdefects in sheep using recombinant human bone morphogenic protein Clin. Orthop. Rel. Res. 293:317-326;1993

Gombotz, W. R., Pankey, S. C., Bouchard, L. S., Stimulation of bone healing by transforming growth factor-beta 1 released from polymeric or ceramic implants. J.appl. Biomater. 5:141-150;1994

Hong., L., Tabata, Y., Miyamoto, S., Yamamoto, M., Yamada, K., Hashimoto, N., and Ikada, Y. Bone regeneration at rabbit skull defects treated with transforming growth factor-β1 incorporated into hydrogels with different levels of biodegradability. J. Neurosurg. 92:315-325;2000

Kehat, I., Kenyagin-Karsenti D., Snir, M., Segev, H., Amit M., Gepstein, A., Livne, E., Binah, O., Itskovitz-Eldor J., and Gepstein, L. Human embryonic stem cells can differentiate into myocytes with structural and functional properties of cardiomyocytes. J. Cli. Invest. 108:407-414, 2001

Kenley, R. A., Yim, K., Abrams, J. Biotechnology and bone graft substitutes. Pharm. Res. 10:1393-1401; 1993

Lee, S. C., Shea, M., Battle, M. A., Kozitza, K., Ron, E., Turek, T., Schaub, R. G., and Hayes, W. C. Healing of large segmental defects in rat femurs is aided by RhBMP-2 in PLGA matrix. J. Biomed. Mat. Res. 28:1149-1156;1994.

Lind, M., Schumacher, B., Soballe, K., Keller, J., Melsen, F. and Bunger, C. Transforming growth factorβenhances fracture healing in rabbit tibia. Acta Orthop. Sacnd. 64:553-556;1993

Lind, M., Soren, O., Nguyen, T., Ongpipattannkul, Soballe, K.and B., Bunger, C. Transforming growth factor beta enhances fixation and bone ongrowth of ceramic coated implants. J. Orthop. Res. 14:343-350;1996.

Lind, M., Soren, O., Nguyen, T., Ongpipattannkul, B., Bunger, C. and Soballe, K. Transforming growth factor beta stimulates bone ongrowth to hydroxyapatite coated implants in dogs. Acta orthop. Scand. 67:611-616;1996

Moxham, J. P., Kibblewhite, D. J., Dvorak, M., Perey, B., Tencer, A. F., Bruce, G., and Strong M. TGF-β1 forms functionally normal bone in a segmental sheep tibial diaphyseal defect. J. Otolaryngol. 25:388-392;1996

Robey, P. G., Young, M. F., Flanders, K. C., Roche, N. S., Kondaiah, P., Reddi, A. H., Termine, J. D., Sporn, M. B., Roberts, A. B., Osteoblasts synthesize and respond to transforming growth factor-type β(TGF-β) in vitro. J.Cell Biol. 105:457-463; 1987

Robinson, D. and Nevo, Z. Articular cartilage chondrocytes are more advantageous for generating hyaline-like cartilage than mesnchymal cells isolated from microfractures repairs. Cell Tiss. Banking. 2:23-30, 2001

Schmitz, J. P., and Hollinger, J. O. The critical size defect as an experimental model for craniomandibular nonunions. Clin. Orthop. Rel. Res. 205:299-308;1986

Sherris, D., Murakami, C. S., Larrabee, Jr., W. F., and Bruce, A. G. Mandibular reconstruction with tarnsforming growth factor-β1. Laryngoscope. 108:368-372;1998

Schulinder, M., Yanuka, O., Itskovitz-Eldor J., Melton, D. A., and Benvenisty N. Effects of eight growth factors on the differentiation of cells derived from human embryonic stem cells. PNAS 97:11307-11312, 2000

Tabata, Y., Yamada, K., Miyamoto, S., Nagata, I., Kikuchi, H., Ayoama, I., Tamura, M. and Ikada, Y. Bone regenerationby fibroblast growth facto complexed with biodegradable hydrogels. Biomaterials 19:807-815; 1998 Thomson, J. A., Iskovitz-Eldor J., Shapiro, S. S., Waknitz, M. A., Swiergiel, J. J., Marshall V. S., and Jones J. M. Embryonic stem cell lines derived from human blasticytes. Science 282:1145-1147, 1998.

Toung, J. S., Griffin, A., Ogle, R. C., and Lindsay, W. H. Repair of nasal defects using collagen gels containing insulin-like growth factor 1. Laryngoscope 108:1654-1658;1998

Yamada, K., Tabata, Y., Yamamoto, K., Potential efficacy of basic fibroblast growth factor incorporated in biodegradable hydrogel for skull bone regeneration. J. Neurosurg. 86:871-875:1997

Yamamoto, M., Tabata, Y., Hong, L., Miyamoto, S., Hashimoto, N., and Ikada, Y. Bone regeneration by transforming growth factor β1 released from a biodegradable hydrogel. J. Control. Rel.64:133-142;2000

Yamamoto, M., Tabata, Y., Ikada, Y. Ectopic bone formation induced by biodegradable hydrogels incorporating bone morphogenic protein. J.Biomaer.Sci.Polym.Ed. 9:439-458; 1998

Yasko, A. W., Lane, J. M., Fellinger, E. J., Rosen, V., Wozney, J. M., Wang, E. The healing of segmental bone defects, induced by recombinant human bone morphogenic protein (rhBMP-2). J.Bone Jn. Surg. 74-A:659-670;1992

Zambonin, G., Camerino, C., Greco, G., Patella, V.,Moretti, B., and Grano, M. Hydroxyapatite coated with hepatocyte growth factor (HGF) stimulates human osteoblasts in vitro. Bone Jt. Surg. 82-B:457-460;2000

What is claimed is:

1. A method of repairing a long bone selected from the group consisting of a femur, a tibia, a humerus and a radius having a defect, the method comprising:
(a) using an external mechanical fixating device for mechanically fixating the long bone or portions thereof; and
(b) filling the defect with a biodegradable cross-linked acidic gelatin designed to promote osteointegration and awaiting for osteointegration to take place.

2. The method of claim 1, further comprising, prior to said fixating or said filling, reshaping the defect.

3. The method of claim 1, wherein (a) precedes (b).

4. The method of claim 1, wherein (b) precedes (a).

5. The method of claim 1, further comprising unfixating the long bone or portions thereof following osteointegration.

6. The method of claim 1, wherein said external mechanical fixating device is a cast.

7. The method of claim 1, wherein said external mechanical fixating device is a bone securing device.

8. The method of claim 1, wherein said biodegradable cross-linked acidic gelatin includes at least one bone growth-promoting agent impregnated therein.

9. The method of claim 8, wherein said at least one bone growth promoting agent is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

10. The method of claim 8, wherein said at least one bone growth-promoting agent comprises at least one cell type expressing and secreting at least one growth factor.

11. The method of claim 10, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

12. The method of claim 1, wherein said biodegradable cross-linked acidic gelatin includes osteoprogenitor cells impregnated therein.

13. The method of claim 12, wherein said osteoprogenitor cells include embryonic stem cells.

14. The method of claim 8, wherein said biodegradable cross-linked acidic gelatin further includes osteoprogenitor cells impregnated therein.

15. The method of claim 14, wherein said osteoprogenitor cells include embryonic stem cells.

16. The method of claim 1, wherein said biodegradable cross-linked acidic gelatin includes at least one drug impregnated therein.

17. The method of claim 16, wherein said at least one drug is selected from the group consisting of an antibiotic agent, a vitamin and an anti-inflammatory agent.

18. The method of claim 17, wherein said antibiotic is selected from the group consisting of an aminoglycoside, a penicillin, a cephalosporin, a semi-synthetic penicillin, and a quinoline.

19. The method of claim 1, wherein said biodegradable cross-linked acidic gelatin includes at least one bone growth promoting agent attached thereto.

20. The method of claim 19, wherein said at least one bone growth promoting agent is selected from the group consisting of an insulin-like growth factor-1 (IGF-1), a transforming growth factory-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

21. The method of claim 19, wherein said at least one bone growth-promoting agent comprises at least one cell type expressing and secreting at least one growth factor.

22. The method of claim 21, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

23. The method of claim 1, wherein said biodegradable cross-linked acidic gelatin includes osteoprogenitor cells attached thereto.

24. The method of claim 23, wherein said osteoprogenitor cells include embryonic stem cells.

25. The method of claim 1, wherein said biodegradable cross-linked acidic gelatin includes at least one drug attached thereto.

26. The method of claim 25, wherein said at least one drug is selected from the group consisting of an antibiotic agent, a vitamin and an anti-inflammatory agent.

27. The method of claim 26, wherein said antibiotic agent is selected from the group consisting of an aminoglycoside, a penicillin, a cephalosporin, a semi-synthetic penicillin, and a quinoline.

28. The method of claim 1, wherein said biodegradable cross-linked acidic gelatin has electrostatic binding properties.

29. The method of claim 1, wherein said biodegradable cross-linked acidic gelatin includes at least one bone degradation inhibitor impregnated therein.

30. The method of claim 29, wherein said at least one bone degradation inhibitor is selected from the group consisting of a collagenase inhibitor, a gelatinase inhibitor, a stromeylsin inhibitor and a plasminogen inhibitor.

31. The method of claim 1, wherein said biodegradable cross-linked acidic gelatin is adapted for sustained release of a therapeutically active agent.

32. The method of claim 1, wherein said biodegradable cross-linked acidic gelatin includes at least one bone degradation inhibitor attached thereto.

33. The method of claim 32, wherein said at least one bone degradation inhibitor is selected from the group consisting of a collagenase inhibitor, a gelatinase inhibitor, a stromeylsin inhibitor and a plasminogen inhibitor.

34. The method of claim 1, wherein the defect is a result of a condition selected from the group consisting of a traumatic injury, a surgery, a birth defect, a developmental defect, aging and a disease.

35. A kit for repairing a long bone selected from the group consisting of a femur, a tibia, a humerus and a radius having a defect, the kit comprising:
(a) an external mechanical fixating device for fixating the long bone or portions thereof; and
(b) a filler for filling the defect, said filler including a biodegradable cross-linked acidic gelatin.

36. The kit of claim 35, wherein said biodegradable cross-linked acidic gelatin includes at least one bone growth-promoting agent impregnated therein.

37. The kit of claim 36, wherein said at least one bone growth promoting agent is selected from the group consisting of an insulin-like growth factor-1 (IGF-1), a transforming growth factory-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

38. The kit of claim 36, wherein said at least one bone growth-promoting agent comprises at least one cell type expressing and secreting at least one growth factor.

39. The kit of claim 38, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

40. The kit of claim 35, wherein said biodegradable cross-linked acidic gelatin includes osteoprogenitor cells impregnated therein.

41. The kit of claim 40, wherein said osteoprogenitor cells include embryonic stem cells.

42. The kit of claim 36, wherein said biodegradable cross-linked acidic gelatin further includes osteoprogenitor cells impregnated therein.

43. The kit of claim 42, wherein said osteoprogenitor cells include embryonic stem cells.

44. The kit of claim 35, wherein said biodegradable cross-linked acidic gelatin includes at least one drug impregnated therein.

45. The kit of claim 44, wherein said at least one drug is selected from the group consisting of an antibiotic agent, a vitamin and an anti-inflammatory agent.

46. The kit of claim 45, wherein said antibiotic agent is selected from the group consisting of an aminoglycoside, a penicillin, a cephalosporin, a semi-synthetic penicillin, and a quinoline.

47. The kit of claim 35, wherein said biodegradable cross-linked acidic gelatin includes at least one bone growth promoting agent attached thereto.

48. The kit of claim 47, wherein said at least one bone growth promoting agent is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

49. The kit of claim 47, wherein said at least one bone growth-promoting agent comprises at least one cell type expressing and secreting at least one growth factor.

50. The kit of claim 49, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

51. The kit of claim 35, wherein said biodegradable cross-linked acidic gelatin includes osteoprogenitor cells attached thereto.

52. The kit of claim 51, wherein said osteoprogenitor cells include embryonic stem cells.

53. The kit of claim 35, wherein said biodegradable cross-linked acidic gelatin includes at least one drug attached thereto.

54. The kit of claim 53, wherein said at least one drug is selected from the group consisting of an antibiotic agent, a vitamin and an anti-inflammatory agent.

55. The kit of claim 54, wherein said antibiotic is selected from the group consisting of an aminoglycoside, a penicillin, a cephalosporin, a semi-synthetic penicillin, and a quinoline.

56. The kit of claim 35, wherein said biodegradable cross-linked acidic gelatin has electrostatic binding properties.

57. The kit of claim 35, wherein said biodegradable cross-linked acidic gelatin includes at least one bone degradation inhibitor impregnated therein.

58. The kit of claim 57, wherein said at least one bone degradation inhibitor is selected from the group consisting of a collagenase inhibitor, a gelatinase inhibitor, a stromeylsin inhibitor and a plasminogen inhibitor.

59. The kit of claim 47, wherein said biodegradable cross-linked acidic gelatin is adapted for sustained release of a therapeutically active agent.

60. The kit of claim 35, wherein said biodegradable cross-linked acidic gelatin includes at least one bone degradation inhibitor attached thereto.

61. The kit of claim 60, wherein said at least one bone degradation inhibitor is selected from the group consisting of a collagenase inhibitor, a gelatinase inhibitor, a stromeylsin inhibitor and a plasminogen inhibitor.

62. The kit of claim 35, wherein said external mechanical fixating device is a bone-securing device.

63. The kit of claim 35, wherein the defect is a result of a medical condition selected from the group consisting of a traumatic injury, a surgery, a birth defect, a developmental defect, aging and a disease.

64. A method of repairing a long bone having a defect, the method comprising:
(a) mechanically fixating the long bone or portions thereof; and
(b) filling the defect with a biodegradable hydrogel containing osteoprogenitor cells and awaiting for osteointegration to take place.

65. The method of claim 64, further comprising, prior to said fixating or said filling, reshaping the defect.

66. The method of claim 64, wherein (a) precedes (b).

67. The method of claim 64, wherein (b) precedes (a).

68. The method of claim 64, further comprising unfixating the long bone or portions thereof following osteointegration.

69. The method of claim 64, wherein said mechanically fixating the long bone or portions thereof is effected by an external mechanical fixating device.

70. The method of claim 69, wherein said external mechanical fixating device is a cast.

71. The method of claim 69, wherein said external mechanical fixating device is a bone securing device.

72. The method of claim 64, wherein said osteoprogenitor cells comprise embryonic stem cells.

73. The method of claim 64, wherein said biodegradable hydrogel comprises a cross-linked polymer.

74. The method of claim 73, wherein said polymer is an acidic protein.

75. The method of claim 74, wherein said protein is an acidic gelatin.

76. The method of claim 64, wherein said biodegradable hydrogel includes at least one bone growth-promoting agent impregnated therein.

77. The method of claim 76, wherein said at least one bone growth promoting agent is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

78. The method of claim 76, wherein said at least one bone growth-promoting agent comprises at least one cell type expressing and secreting at least one growth factor.

79. The method of claim 78, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

80. The method of claim 64, wherein said biodegradable hydrogel includes at least one drug impregnated therein.

81. The method of claim 80, wherein said at least one drug is selected from the group consisting of an antibiotic agent, a vitamin and an anti-inflammatory agent.

82. The method of claim 81, wherein said antibiotic is selected from the group consisting of an aminoglycoside, a penicillin, a cephalosporin, a semi-synthetic penicillin, and a quinoline.

83. The method of claim 73, wherein said biodegradable hydrogel includes at least one bone growth promoting agent attached to said polymer.

84. The method of claim 83, wherein said at least one bone growth promoting agent is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

85. The method of claim 83, wherein said at least one bone growth-promoting agent comprises at least one cell type expressing and secreting at least one growth factor.

86. The method of claim 85, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

87. The method of claim 73, wherein said biodegradable hydrogel includes at least one drug attached to said polymer.

88. The method of claim 87, wherein said at least one drug is selected from the group consisting of an antibiotic agent, a vitamin and an anti-inflammatory agent.

89. The method of claim 88, wherein said antibiotic agent is selected from the group consisting of an aminoglycoside, a penicillin, a cephalosporin, a semi-synthetic penicillin, and a quinoline.

90. The method of claim 64, wherein the long bone is selected from the group consisting of tibia, femur, humerus, radius, ulna, fibula, carpals, metacarpals, phalanges, tarsals, and metatarsals.

91. The method of claim 64, wherein said biodegradable hydrogel has electrostatic binding properties.

92. The method of claim 76, wherein said biodegradable hydrogel includes at least one bone degradation inhibitor impregnated therein.

93. The method of claim 92, wherein said at least one bone degradation inhibitor is selected from the group consisting of a collagenase inhibitor, a gelatinase inhibitor, a stromeylsin inhibitor and a plasminogen inhibitor.

94. The method of claim 64, wherein said biodegradable hydrogel is adapted for sustained release of a therapeutically active agent.

95. The method of claim 73, wherein said biodegradable hydrogel includes at least one bone degradation inhibitor attached to said polymer.

96. The method of claim 95, wherein said at least one bone degradation inhibitor is selected from the group consisting of a collagenase inhibitor, a gelatinase inhibitor, a stromeylsin inhibitor and a plasminogen inhibitor.

97. The method of claim 64, wherein the defect is a result of a condition selected from the group consisting of a traumatic injury, a surgery, a birth defect, a developmental defect, aging and a disease.

98. The method of claim 64, wherein said long bone is selected from the group consisting of a femur, a tibia, a humerus and a radius.

99. A kit for repairing a long bone having a defect, the kit comprising:
(a) a mechanical fixating device for fixating the long bone or portions thereof; and
(b) a filler for filling the defect, said filler including a biodegradable hydrogel containing osteoprogenitor cells.

100. The kit of claim 99, wherein said osteoprogenitor cells comprise embryonic stem cells.

101. The kit of claim 99, wherein said mechanical fixating device is an external mechanical fixating device.

102. The kit of claim 99, wherein said biodegradable hydrogel comprises a cross-linked polymer.

103. The kit of claim 102, wherein said polymer is an acidic protein.

104. The kit of claim 103, wherein said acidic protein is an acidic gelatin.

105. The kit of claim 99, wherein said biodegradable scaffold includes at least one bone growth-promoting agent impregnated therein.

106. The kit of claim 105, wherein said at least one bone growth promoting agent is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

107. The kit of claim 105, wherein said at least one bone growth-promoting agent comprises at least one cell type expressing and secreting at least one growth factor.

108. The kit of claim 107, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

109. The kit of claim 99, wherein said biodegradable hydrogel includes at least one drug impregnated therein.

110. The kit of claim 109, wherein said at least one drug is selected from the group consisting of an antibiotic agent, a vitamin and an anti-inflammatory agent.

111. The kit of claim 110, wherein said antibiotic agent is selected from the group consisting of an aminoglycoside, a penicillin, a cephalosporin, a semi-synthetic penicillin, and a quinoline.

112. The kit of claim 102, wherein said biodegradable hydrogel includes at least one bone growth promoting agent attached to said polymer.

113. The kit of claim 112, wherein said at least one bone growth promoting agent is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

114. The kit of claim 112, wherein said at least one bone growth-promoting agent comprises at least one cell type expressing and secreting at least one growth factor.

115. The kit of claim 114, wherein said at least one growth factor is selected from the group consisting of an insulin-like growth factor-1 (IGF-1), a transforming growth factor-β(TGF-β), a basic fibroblast growth factor (bFGF), a bone morphogenic protein (BMP), a cartilage-inducing factor-A, a cartilage-inducing factor-B, an osteoid-inducing factor, a collagen growth factor and osteogenin.

116. The kit of claim 102, wherein said biodegradable hydrogel includes at least one drug attached to said polymer.

117. The kit of claim 116, wherein said at least one drug is selected from the group consisting of an antibiotic agent, a vitamin and an anti-inflammatory agent.

118. The kit of claim 117, wherein said antibiotic is selected from the group consisting of an aminoglycoside, a penicillin, a cephalosporin, a semi-synthetic penicillin, and a quinoline.

119. The kit of claim 99, wherein the long bone is selected from the group consisting of tibia, femur, humerus, radius, ulna, fibula, carpals, metacarpals, phalanges, tarsals, and metatarsals.

120. The kit of claim 99, wherein said biodegradable hydrogel has electrostatic binding properties.

121. The kit of claim 112, wherein said biodegradable hydrogel includes at least one bone degradation inhibitor impregnated therein.

122. The kit of claim 121, wherein said at least one bone degradation inhibitor is selected from the group consisting of a collagenase inhibitor, a gelatinase inhibitor, a stromeylsin inhibitor and a plasminogen inhibitor.

123. The kit of claim 112, wherein said biodegradable hydrogel is adapted for sustained release of a therapeutically active agent.

124. The kit of claim 102, wherein said biodegradable hydrogel includes at least one bone degradation inhibitor attached to said polymer.

125. The kit of claim 124, wherein said at least one bone degradation inhibitor is selected from the group consisting of a collagenase inhibitor, a gelatinase inhibitor, a stromeylsin inhibitor and a plasminogen inhibitor.

126. The kit of claim 99, wherein said mechanical fixating device is a bone-securing device.

127. The kit of claim 99, wherein the defect is a result of a medical condition selected from the group consisting of a traumatic injury, a surgery, a birth defect, a developmental defect, aging and a disease.
